(19) United States
(12) Patent Application Publication
(10) Pub. No.: US 2023/0313111 A1
(43) Pub. Date: Oct. 5, 2023

(54) BIOREACTORS CONFIGURED FOR UV STERILIZATION, AND METHODS OF USING UV STERILIZATION IN BIOPROCESSES

(71) Applicant: ARCOLOGY INC. DBA BIOSPHERE, Culver City, CA (US)

(72) Inventor: Brian HELIGMAN, North Potomac, MD (US)

(21) Appl. No.: 18/121,153

(22) Filed: Mar. 14, 2023

Related U.S. Application Data
(60) Provisional application No. 63/327,258, filed on Apr. 4, 2022.

(51) Int. Cl. C12M 1/12; C12M 1/24; C12M 1/00; C12M 1/107

(52) U.S. Cl. CPC C12M 37/02; C12M 23/08; C12M 41/06; C12M 23/34; C12M 23/36; C12M 23/22

(57) ABSTRACT

In some variations, a UV-sterilizable bioreactor system comprises: a chamber configured to carry out a reaction; a component configured for introducing a gas into the chamber; and one or more UV light sources configured to expose ultraviolet light to surfaces within the chamber. Some variations provide a method of cleaning and sterilizing a bioreactor, comprising: providing a bioreactor system with a chamber to carry out a reaction, a component for introducing a gas into the chamber, and one or more UV light sources to expose ultraviolet light to chamber surfaces; cleaning the chamber; and exposing the chamber to UV radiation to sterilize the chamber. The disclosed technology is a fundamental advance in the field of sparged bioreactors, for many types of commercial fermentations. The bioreactor may incorporate materials that are expressly incompatible with steam sterilization, which allows for the use of lower-cost materials, among many other benefits from UV sterilization.

### PRIORITY DATA

This patent application claims priority to U.S. Provisional Patent App. No. 63/327,258 entitled “UV BIOREACTOR”, filed on Apr. 4, 2022, which is hereby incorporated by reference herein.

### FIELD

The present invention generally relates to sterilization of bioreactors, the design of bioreactors enabling efficient sterilization, and methods of sterilizing bioreactors and other bioprocess components.

### BACKGROUND

In fermentation, feedstocks are transformed into higher-value products using biological systems. Advanced fermentation processes can be carried out within bioreactors, physical systems which create an optimal environment for the biological system by combining solid, liquid, or gaseous feedstocks within a controlled environment. In modern embodiments of bioreactors, this often entails the combination of at least one liquid and at least one gaseous feedstock.

For biological production to be effective, it is important that the presence of undesirable biological agents (known as adventitious organisms) is limited to the greatest extent possible. This is critical for ensuring the highest possible fraction of feedstock is converted into the desired product or products of interest, rather than alternative and undesirable products. In modern bioreactor systems, the presence of adventitious organisms is reduced by thoroughly cleaning the reactor system, critically utilizing steam. This process of reducing adventitious population is often deemed sterilization, although in some cases a fraction of adventitious organism remains within the system.

It is well-understood that radiation inactivates microbial organisms. In the prior art, adventitious agents have been removed from a bioreactor system through the use of high-energy gamma radiation in a controlled environment. The ionizing nature of gamma radiation allows for uniform distribution of the sterilizing radiation throughout the entirety of the bioreactor's volume. However, the ability of gamma radiation to penetrate structural features substantially complicates the sterilization process, as this radiation poses a substantial threat to human health. For that reason, bioreactor sterilization via gamma irradiation occurs in centralized manufacturing facilities, and these vessels are traditionally disposed of after a single use.

### SUMMARY

In some variations, the present invention provides a UV-sterilizable bioreactor system comprising:
(a) a chamber configured to carry out a reaction;
(b) a component configured for introducing a gas into the chamber; and
(c) one or more UV light sources configured to expose ultraviolet light to surfaces within the chamber.

In preferred embodiments, the chamber is configured to maintain a sterile boundary with the environment.

In some embodiments, the component for introducing a gas is a gas sparger. In some embodiments, the component for introducing a gas is a membrane. The gas may be air, oxygen, syngas, hydrogen, carbon monoxide, methane, natural gas, or a combination thereof. In typical embodiments, the gas is oxygen, air, oxygen-enriched air, or oxygen-depleted air.

In preferred embodiments, the component for introducing a gas is UV-sterilizable. In some embodiments, the component for introducing a gas contains a UV-transparent material. The UV-transparent material may contain a UV-transparent polymer, a UV-transparent ceramic, a UV-transparent glass, or a combination thereof. A UV-transparent polymer may be selected from the group consisting of polyacrylates, silicones, fluoropolymers, and combinations thereof. Fluoropolymers may be selected from the group consisting of polyvinylidene fluoride, polytetrafluoroethylene, perfluoroalkoxy alkanes, and combinations thereof. A UV-transparent ceramic may be selected from the group consisting of quartz, fused silica, borosilicates, and combinations thereof.

In some embodiments, the UV-sterilizable bioreactor system further comprises one or more conduits (e.g., valves) configured to feed an input material into the chamber and/or to withdraw an output material out of the chamber. Preferably, at least one of the one or more conduits is UV-sterilizable. In some embodiments, there are 2, 3, or more conduits, wherein each of the conduits is UV-sterilizable.

In some embodiments, one or more UV light sources are situated within the chamber. One or more UV light sources may be permanently situated within the chamber. Alternatively, or additionally, one or more UV light sources may be reversibly situated within the chamber.

In some embodiments, one or more UV light sources are situated within UV-transparent wells that are disposed within the chamber. The UV-transparent wells may be fabricated from a UV-transparent material containing a UV-transparent polymer, a UV-transparent ceramic, a UV-transparent glass, or a combination thereof. A UV-transparent polymer may be selected from the group consisting of polyacrylates, silicones, fluoropolymers, and combinations thereof. Fluoropolymers may be selected from the group consisting of polyvinylidene fluoride, polytetrafluoroethylene, perfluoroalkoxy alkanes, and combinations thereof. A UV-transparent ceramic may be quartz. A UV-transparent glass may be fused silica, borosilicates, or a combination thereof.

In some embodiments, at least some (including at least one) of the one or more UV light sources are external to the chamber. In certain embodiments, all of the one or more UV light sources are external to the chamber.

In some embodiments, the chamber has walls fabricated from a metal, a metal alloy, a polymer, a ceramic, a composite material, glass, concrete, or a combination thereof. In certain embodiments, the metal is aluminum, copper, nickel, silver, or a combination thereof. In certain embodiments, the metal alloy is carbon steel or stainless steel. In certain embodiments, the polymer is selected from the group consisting of polyolefins, polyacrylates, polycarbonates, fluoropolymers, silicones, and combinations thereof. In certain embodiments, the composite material is a polymer reinforced with glass fibers. In certain embodiments, the polymer is selected from high-density polyethylene, polypropylene, polycarbonate, or a combination thereof. The polymer may be selected from the group consisting of poly(methyl methacrylate), polyvinylidene fluoride, hexafluoropropylene-tetrafluoroethylene copolymers, perfluoroether-tetrafluoroethylene copolymers, and combinations thereof. The chamber walls may also be coated with an antimicrobial surface, such as copper, silver, or a nanostructured coating, for example.

In some embodiments, the chamber has UV-transparent chamber walls containing a UV-transparent material. The UV-transparent material may contain a UV-transparent polymer, a UV-transparent ceramic (e.g., quartz), a UV-transparent glass (e.g., fused silica and/or borosilicates), or a combination thereof. A UV-transparent polymer may be selected from the group consisting of polyacrylates (e.g., poly(methyl methacrylate)), silicones, fluoropolymers, and combinations thereof. Fluoropolymers may be selected from the group consisting of polyvinylidene fluoride, hexafluoropropylene-tetrafluoroethylene copolymers, perfluoroether-tetrafluoroethylene copolymers, poly(ethene-co-tetrafluoroethene), or a combination thereof.

In certain embodiments, the chamber has UV-reflective chamber walls containing, or internally coated with, a UV-reflective material. The UV-reflective material may be selected from aluminum, stainless steel, polytetrafluoroethylene, or a combination thereof, for example.

In certain embodiments, the chamber is configured with a chamber top that is not sealed from the environment, but the chamber top is UV-sterilizable to form a sterile barrier with the environment.

The one or more UV light sources may be configured to expose ultraviolet light to at least 80%, at least 85%, at least 90%, at least 95%, or at least 99% of the total surface area within the chamber including its internal components.

The one or more UV light sources may be configured to expose ultraviolet light to at least 80%, at least 85%, at least 90%, at least 95%, or at least 99% of the total volume of the chamber.

In some embodiments, the one or more UV light sources each have a UV wavelength selected from about 100 nm to about 400 nm. In certain embodiments, the UV wavelength is selected from about 220 nm to about 300 nm, for at least one of the one or more UV light sources, such as for all of the UV light sources.

The one or more UV light sources may each be selected from the group consisting of UV light-emitting diodes, UV mercury lamps, UV xenon lamps, and UV krypton lamps, for example.

In some embodiments, the bioreactor system contains an impeller situated within the chamber, wherein the impeller is preferably UV-sterilizable. In other embodiments, the chamber does not contain an impeller.

In some embodiments, the UV-sterilizable bioreactor system further comprises a filtration unit configured to filter an input material before being fed into the chamber. In certain embodiments, the filtration unit is UV-sterilizable.

In some embodiments, the UV-sterilizable bioreactor system contains a bioreactor sensor situated within the chamber. The bioreactor sensor may be configured to detect or measure a bioreactor parameter selected from the group consisting of pH, temperature, oxygen, carbon dioxide, foaming, mixing, cell density, feed-substrate concentration, reaction-intermediate concentration, and product concentration, for example. There may be multiple bioreactor sensors situated within the chamber. In some embodiments, the bioreactor sensor is configured to transmit a wireless signal.

In some embodiments, the bioreactor sensor is UV-sterilizable. The bioreactor sensor may be configured with a UV optical waveguide for sterilizing the bioreactor sensor. The UV optical waveguide may be a UV optical fiber, for example.

In some embodiments, the bioreactor sensor is disposed within or through a probe port, wherein the probe port is UV-sterilizable. In some embodiments, the bioreactor sensor is contained in a UV-sterilizable housing that is situated within the chamber. The UV-sterilizable housing may contain a UV-transparent material, such as a UV-transparent polymer, a UV-transparent ceramic, a UV-transparent glass, or a combination thereof. A UV-transparent polymer may be selected from the group consisting of polyacrylates, silicones, fluoropolymers, and combinations thereof. An exemplary polyacrylate is poly(methyl methacrylate). Fluoropolymers may be selected from the group consisting of polyvinylidene fluoride, hexafluoropropylene-tetrafluoroethylene copolymers, perfluoroether-tetrafluoroethylene copolymers, poly(ethene-co-tetrafluoroethene), and combinations thereof. An exemplary UV-transparent ceramic is quartz. Exemplary UV-transparent glasses include fused silica, borosilicates, or a combination thereof.

In some embodiments, the bioreactor system contains a clean-in-place arm situated within the chamber. The clean-in-place arm is preferably UV-sterilizable.

Some variations generally provide a radiation-sterilizable bioreactor system comprising:
(a) a chamber configured to carry out a reaction;
(b) a component configured for introducing a gas into the chamber; and
(c) one or more light sources configured to expose radiation to surfaces within the chamber, wherein the radiation has a wavelength longer than 1 nm but less than 1 mm.

In preferred embodiments, the one or more light sources are configured to expose UV radiation, visible light, IR radiation, or a combination thereof to the surfaces within the chamber.

Some variations provide a method of cleaning and sterilizing a bioreactor, the method comprising:
(i) providing a UV-sterilizable bioreactor system comprising: (a) a chamber configured to carry out a reaction; (b) a component configured for introducing a gas into the chamber; and (c) one or more UV light sources configured to expose ultraviolet light to surfaces within the chamber;
(ii) cleaning the chamber; and
(iii) exposing the chamber to UV radiation to sterilize the chamber.

In some methods, the UV-sterilizable bioreactor system further comprises one or more conduits (e.g., valves) configured to feed an input material into the chamber and/or to withdraw an output material out of the chamber. In certain methods, at least one of the conduits is UV-sterilizable.

In some methods, one or more UV light sources, such as all of the UV light sources, are situated within the chamber. In certain methods, one or more UV light sources, such as all of the UV light sources, are permanently situated within the chamber. In certain methods, one or more UV light sources, such as all of the UV light sources, are reversibly situated within the chamber.

In some methods, one or more UV light sources, such as all of the UV light sources, are situated within UV-transparent wells that are disposed within the chamber.

In some methods, at least some of the one or more UV light sources are external to the chamber. In certain methods, all of the one or more UV light sources are external to the chamber.

In some methods, the chamber has walls fabricated from a metal, a metal alloy, a polymer, a ceramic, a composite material, glass, concrete, or a combination thereof.

In some methods, the chamber has UV-transparent chamber walls containing a UV-transparent material.

In some methods, the chamber has UV-reflective chamber walls containing, or internally coated with, a UV-reflective material.

In certain methods, the chamber is configured with a chamber top that is not sealed from the environment, wherein the chamber top is UV-sterilizable to form a sterile barrier with the environment.

In some methods, an impeller is situated within the chamber, wherein the impeller is UV-sterilizable.

In some methods, the UV-sterilizable bioreactor system further comprises a filtration unit configured to filter an input material before being fed into the chamber. In certain methods, the filtration unit is UV-sterilizable.

In some methods, a bioreactor sensor is situated within the chamber. The bioreactor sensor may be configured to detect or measure a bioreactor parameter selected from the group consisting of pH, temperature, oxygen, carbon dioxide, foaming, mixing, cell density, feed-substrate concentration, reaction-intermediate concentration, and product concentration. During operation, the bioreactor sensor may actually detect or measure a bioreactor parameter. The bioreactor sensor may be configured to transmit a wireless signal during or after sensor measurement.

In some methods, the bioreactor sensor is UV-sterilizable. The bioreactor sensor may be configured with a UV optical waveguide for sterilizing the bioreactor sensor. Alternatively, or additionally, the bioreactor sensor is contained in a UV-sterilizable housing that is situated within the chamber. The UV-sterilizable housing may contain a UV-transparent material. Alternatively, or additionally, the bioreactor sensor may be disposed within or through a probe port, wherein the probe port is UV-sterilizable.

In some methods, the bioreactor contains a clean-in-place arm situated within the chamber, wherein the clean-in-place arm is UV-sterilizable.

In some methods, step (ii) utilizes a cleaning agent selected from the group consisting of hot water, an alkaline detergent, sodium hydroxide, sodium percarbonate, an acidic detergent, phosphoric acid, peracetic acid, isopropanol, ethanol, sodium hypochlorite, and combinations thereof. Other cleaning agents may be utilized instead of, or in addition to, these chemicals.

In some methods, step (iii) utilizes a sterilization energy per unit total area in the chamber of about 50 mJ/cm² or more.

In some methods, step (iii) utilizes a total UV power output from about 0.5 mW/cm² to about 10000 mW/cm².

In some methods, step (iii) utilizes a total UV power capacity from about 0.1 W/m³ to about 10 W/m³.

In some methods, step (iii) utilizes a UV sterilization time from about 60 seconds to about 2 hours.

In some methods, step (iii) exposes the UV radiation to at least 80% of total surface area within the chamber including its internal components. Step (iii) may expose the UV radiation to at least 85%, at least 90%, at least 95%, or at least 99% (including 100%) of total surface area within the chamber including its internal components.

In some methods, step (iii) exposes the UV radiation to at least 80% of total volume of the chamber. Step (iii) may expose the UV radiation to at least 85%, at least 90%, at least 95%, or at least 99% (including 100%) of total volume of the chamber.

In some methods, step (iii) utilizes a UV wavelength selected from about 100 nm to about 400 nm, such as a UV wavelength is selected from about 220 nm to about 300 nm.

In some methods, the one or more UV light sources are each selected from the group consisting of UV light-emitting diodes, UV mercury lamps, UV xenon lamps, and UV krypton lamps.

In various methods, step (iii) is effective to reach a 4-log reduction in adventitious microorganisms present prior to step (iii). In some methods, step (iii) is effective to reach a 6-log reduction in adventitious microorganisms present prior to step (iii). In certain methods, step (iii) is effective to reach a 8-log reduction in adventitious microorganisms present prior to step (iii). In specific methods, step (iii) is effective to reach a 10-log reduction in adventitious microorganisms present prior to step (iii).

The method may further comprise, after step (iii), introducing the gas to the chamber. The gas may be air, oxygen, syngas, hydrogen, carbon monoxide, methane, natural gas, or a combination thereof, for example. The gas may be a mixture of O₂ and N₂ in various concentrations of O₂, above or below 21 vol % O₂.

In preferred methods, the chamber maintains a sterile boundary with the environment.

Some variations generally provide a method of cleaning and sterilizing a bioreactor, the method comprising:
(i) providing a radiation-sterilizable bioreactor system comprising: (a) a chamber configured to carry out a reaction; (b) a component configured for introducing a gas into the chamber; and (c) one or more light sources configured to expose radiation to surfaces within the chamber;
(ii) cleaning the chamber; and
(iii) exposing the chamber to radiation to sterilize the chamber, wherein the radiation has a wavelength longer than 1 nm but less than 1 mm.

### BRIEF DESCRIPTION OF THE DRAWINGS

FIG. 1 is a schematic diagram (side view) of a UV-sterilizable bioreactor system, in some embodiments of the invention.

FIG. 2 is a schematic diagram (isometric view) of a UV-sterilizable bioreactor system, in some embodiments of the invention.

FIG. 3 is a schematic diagram (side view) of a UV-sterilizable bioreactor top with stirrer seal, in some embodiments of the invention.

FIG. 4 is a schematic diagram (top view) of a UV-sterilizable sparger, in some embodiments of the invention.

FIG. 5 is a schematic diagram (isometric view) of a UV-sterilizable sparger, in some embodiments of the invention.

### DETAILED DESCRIPTION OF EMBODIMENTS

The apparatus, structures, methods, systems, and products of the present invention will be described in detail by reference to various non-limiting embodiments.

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying figures.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phrase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phrase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms, except when used in a Markush group. Thus in some embodiments not otherwise explicitly recited, any instance of “comprising” may be replaced by “consisting of” or, alternatively, by “consisting essentially of.”

The present invention is premised on the use of UV light, or other forms of non-ionizing radiation, to effectively sterilize bioreactors. The disclosed technology is a fundamental advance in the field of sparged bioreactors, for many types of commercial fermentations. The bioreactor may incorporate materials that are expressly incompatible with steam sterilization, which allows for the use of lower-cost materials, among many other benefits arising from UV sterilization.

Conventionally, sterile conditions are generated within bioreactor systems using steam sterilization. Such bioreactor systems are evacuated and subsequently pressurized with typically 121° C. steam. The steam environment is maintained until the adventitious microorganisms are inactivated, at which point the steam is removed from the system and bioreactor operation can begin. This process of steam sterilization introduces significant costs to the system operator. First, a significant amount of energy is required to generate the steam, which is both expensive as well as highly carbon-intensive unless renewable energy is used to generate the steam, which adds more cost. Also, designing the bioreactor for steam compatibility introduces significant additional costs and complexities. The present inventor has recognized the problem and the need for better methods of generating sterile operating conditions within a bioreactor environment.

It is known that ultraviolet light can kill or deactivate living organisms. Ultraviolet radiation, mainly UV-C is one of the powerful agents that can alter the normal state of life by inducing a variety of mutagenic and cytotoxic DNA lesions such as cyclobutane-pyrimidine dimers, 6-4 photoproducts, and DNA strand breaks by interfering the genome integrity. See Rastogi et al., “Molecular Mechanisms of Ultraviolet Radiation-Induced DNA Damage and Repair”, Journal of Nucleic Acids, Volume 2010, Article ID 592980, Pages 1-32 (2010), which is hereby incorporated by reference.

In some variations, the present invention provides a UV-sterilizable bioreactor system comprising:
(a) a chamber configured to carry out a reaction;
(b) a component configured for introducing a gas into the chamber; and
(c) one or more UV light sources configured to expose ultraviolet light to surfaces within the chamber.

In this specification, “UV-sterilizable” means that a system or component is capable of being sterilized by UV light, at least to some extent. In this specification, “sterilized”, “sterilize”, “sterilization”, and the like refer to the killing, deactivation, or removal of microorganism to various extents. Sterilization does not mean that absolutely all microorganisms have been killed, deactivated, or removed (which may be referred to as “aseptic”). Certain embodiments may provide aseptic conditions for the system or component being sterilized.

The chamber is preferably a bioreactor chamber configured to carry out a bioreaction, such as fermentation or enzymatic conversion. In this specification, a “bioreactor” is a fermentor that utilizes at least one gas (typically air or oxygen) in the fermentation reaction(s). In other embodiments, the chamber is a reactor chamber configured to carry out an anaerobic bioreaction or a non-biological reaction that does not utilize microorganisms or enzymes.

In preferred embodiments, the chamber is configured to maintain a sterile boundary with the environment. A sterile boundary means that adventitious microorganisms do not penetrate into the bioreactor chamber from the environment. An “adventitious microorganism” is any microorganism (e.g., yeast, bacteria, fungi, mold) that is not the desired microorganism (biocatalyst) for catalyzing the intended reaction in the chamber. Adventitious microorganisms may be referred to as contaminant microorganisms. Adventitious microorganisms can compete with the biocatalyst for resources, introduce undesirable properties, or generate side products within the bioreactor environment.

The gas introduced to the chamber may be a reactant, such as in aerobic or microaerobic fermentation, or may be a catalyst, promoter, reaction-rate moderator, or other reaction agent. The gas introduced to the chamber may be air, oxygen, syngas, hydrogen, carbon monoxide, methane, natural gas, or a combination thereof. In typical embodiments, the gas is oxygen, air, oxygen-enriched air, or oxygen-depleted air.

An external oxygen concentration may be utilized to increase the oxygen content of air beyond the normal 21 vol % O₂ concentration. In various embodiments employing O₂ in fermentation, the O₂ concentration in the gas stream fed to the chamber (e.g., through a sparger) is about, at least about, or at most about 1 vol %, 2 vol %, 5 vol %, 10 vol %, 15 vol %, 20 vol %, 21 vol %, 22 vol %, 25 vol %, 30 vol %, 40 vol %, 50 vol %, 60 vol %, 70 vol %, 80 vol %, 90 vol %, 95 vol %, or 100 vol %, including all intervening ranges.

In some embodiments, the component for introducing a gas is a gas sparger. A sparger may be defined as a component for introducing a gas into the liquid within a bioreactor. Three basic types of spargers are porous spargers, orifice spargers, and nozzle spargers. Spargers are tailored to introduce the desired gas in a controlled manner, resulting in mass transfer between the liquid phase and gas phase, while also introducing mechanical energy into the system.

In some embodiments, the component for introducing a gas is a membrane. These components serve to introduce the gaseous substrate via a semi-permeable membrane, allowing for selective mass transfer of desired components between a gaseous phase and a liquid phase. In some cases, this mass transfer occurs via diffusion within a polymeric matrix, while in other cases this mass transfer occurs via diffusion within pores that are within the membrane itself. In certain embodiments, the gaseous component being transported across the membrane is oxygen.

In some embodiments, the component for introducing a gas is a pipe or tube that is disposed in the volume of the bioreactor and that directly injects a gas into the liquid phase. The pipe or tube may have a single outlet, such as near the bottom of the chamber, near the top of the chamber, or anywhere else in the chamber. The pipe or tube may have multiple outlets, such as a perforated pipe with a plurality of holes, out of which a gas enters the liquid phase.

In some embodiments, the component for introducing a gas is a plate sparger. A plate sparger is typically located at the bottom of the chamber and has a plurality of holes, out of which a gas enters the liquid phase.

In preferred embodiments, the component for introducing a gas is UV-sterilizable. The component (e.g., sparger) for introducing a gas may be UV-sterilizable by being exposed to a UV light source that is external to the component (and internal or external to the chamber). Alternatively, or additionally, the component may be UV-sterilizable by incorporating a UV light source within the component itself.

In some embodiments, the component for introducing a gas contains a UV-transparent material. In this specification, a “UV-transparent material” is not necessarily completely UV-transparent; some absorption of UV radiation may occur. In particular, “UV-transparent” means a sheet of material with 1-millimeter thickness absorbs less than 50%, preferably about 25% or less, more preferably about 10% or less, most preferably about 9%, 8%, 7%, 6%, 5%, 4%, 3%, 2%, 1%, or less, of incident (perpendicular) UV light at a wavelength of interest. The actual material or component need not be 1 millimeter; that thickness is only specified when measuring the UV transmission for purposes of this paragraph. If a wavelength range is used, the UV transmission is averaged over that range. UV transmission can be determined experimentally, for example, using a Perkin Elmer UV-Vis-IR spectrometer. It is noted that UV transmission generally can include regular UV transmission and diffuse UV transmission, both of which can contribute to total UV transmission.

The UV-transparent material (in the component for introducing a gas) may contain a UV-transparent polymer, a UV-transparent ceramic, a UV-transparent glass, or a combination thereof. A UV-transparent polymer may be selected from the group consisting of polyacrylates, silicones, fluoropolymers, and combinations thereof. Fluoropolymers may be selected from the group consisting of polyvinylidene fluoride, polytetrafluoroethylene, perfluoroalkoxy alkanes, and combinations thereof. A UV-transparent ceramic may be selected from the group consisting of quartz, fused silica, borosilicates, and combinations thereof. Silica or borosilicates may be doped to modify their UV transparency.

The number of UV light sources may vary widely. In some embodiments, there is a single UV light source configured to expose ultraviolet light to surfaces within the chamber. In other embodiments, there are multiple UV light sources each configured to expose ultraviolet light to surfaces within the chamber. The number of individual UV light sources can vary widely, such as 1, 2, 3, 4, 5, 6, 7, 8, 9, 10, 25, 50, 100, or more. In certain embodiments employing one or more micro-arrays of UV LED light sources, the number of individual UV light sources can be many hundreds or thousands.

In some embodiments, one or more UV light sources are situated within the chamber. One or more UV light sources may be permanently situated within the chamber. Alternatively, or additionally, one or more UV light sources may be reversibly situated within the chamber. The UV light source(s) may be removed from the chamber in such a manner that sterile conditions are maintained within the chamber.

In some embodiments, one or more UV light sources are situated within UV-transparent wells that are disposed within the chamber. The UV-transparent wells may be fabricated from a UV-transparent material containing a UV-transparent polymer, a UV-transparent ceramic, a UV-transparent glass, or a combination thereof. A UV-transparent polymer may be selected from the group consisting of polyacrylates, silicones, fluoropolymers, and combinations thereof. Fluoropolymers may be selected from the group consisting of polyvinylidene fluoride, polytetrafluoroethylene, perfluoroalkoxy alkanes, and combinations thereof. A UV-transparent ceramic may be quartz. A UV-transparent glass may be fused silica, borosilicates, or a combination thereof. Silica or borosilicates may be doped to modify their UV transparency.

In some embodiments, at least some (including at least one) of the one or more UV light sources are external to the chamber. In certain embodiments, all of the one or more UV light sources are external to the chamber.

Typically, the chamber walls facing the inside of the chamber need to be sterilized, but the outside chamber walls exposed to the environment do not need to be sterilized. In some embodiments, however, it can be beneficial to have at least one external UV light source that exposes UV light to an outside wall, or a selected portion thereof, to kill or deactivate adventitious microorganisms before they might penetration through a small opening into the chamber. For example, in some embodiments, UV LEDs may be continuously utilized during bioreactor operation to prevent introduction of adventitious microorganisms from entering the bioreactor environment.

In some embodiments, one or more UV light sources are configured to provide radiant UV flux to a corner of the chamber, such as a corner defined by part of the chamber wall and an internal component (e.g., chamber lid or agitator shaft). In certain embodiments, discrete UV LED fixtures are utilized for specific components of the bioreactor that would otherwise be inaccessible to a primary UV light source, such as a UV mercury lamp.

In some embodiments, the UV-sterilizable bioreactor system further comprises one or more conduits configured to feed an input material into the chamber and/or to withdraw an output material out of the chamber. Preferably, at least one of the one or more conduits is UV-sterilizable. In some embodiments, there are 2, 3, or more conduits, wherein each of the conduits is UV-sterilizable.

In some embodiments, the chamber has walls fabricated from a metal, a metal alloy, a polymer, a ceramic, a composite material, glass, concrete, or a combination thereof. In certain embodiments, the metal is aluminum. In certain embodiments, the metal alloy is carbon steel or stainless steel. In certain embodiments, the polymer is selected from the group consisting of polyolefins, polyacrylates, polycarbonates, fluoropolymers, silicones, and combinations thereof. In certain embodiments, the composite material is a polymer reinforced with glass fibers. In certain embodiments, the polymer is selected from high-density polyethylene, polypropylene, polycarbonate, or a combination thereof. The polymer may be selected from the group consisting of poly(methyl methacrylate), polyvinylidene fluoride, hexafluoropropylene-tetrafluoroethylene copolymers, perfluoroether-tetrafluoroethylene copolymers, and combinations thereof.

In some embodiments, the chamber has UV-transparent chamber walls containing a UV-transparent material. The UV-transparent material may contain a UV-transparent polymer, a UV-transparent ceramic (e.g., quartz), a UV-transparent glass (e.g., fused silica and/or borosilicates), or a combination thereof. A UV-transparent polymer may be selected from the group consisting of polyacrylates (e.g., poly(methyl methacrylate)), silicones, fluoropolymers, and combinations thereof. Fluoropolymers may be selected from the group consisting of polyvinylidene fluoride, hexafluoropropylene-tetrafluoroethylene copolymers, perfluoroether-tetrafluoroethylene copolymers, poly(ethene-co-tetrafluoroethene), or a combination thereof.

In certain embodiments, the chamber has UV-reflective chamber walls containing, or internally coated with, a UV-reflective material. The UV-reflective material may be selected from aluminum, stainless steel, polytetrafluoroethylene, or a combination thereof, for example.

In certain embodiments, the chamber is configured with a chamber top that is not sealed from the environment, but the chamber top is UV-sterilizable to form a sterile and possibly aseptic barrier with the environment.

The chamber volume may vary widely for a given bioreactor system, including laboratory scale, pilot scale, demonstration scale, and commercial scale. In various embodiments, the chamber volume is about, at least about, or at most about 10 mL, 100 mL, 250 mL, 1 L, 2 L, 5 L, 10 L, 25 L, 50 L, 100 L, 500 L, 1,000 L, 5,000 L, 10,000 L, 50,000 L, 100,000 L, 500,000 L, 1,000,000 L, 2,000,000 L, 5,000,000 L, or greater.

The chamber geometry may also vary. A typical chamber is cylindrical with rounded walls (circular with respect to the vertical axis). A chamber may have rounded walls, flat walls, or a combination thereof. Chamber geometries may generally include cylindrical, tubular, conical, spherical, or rectangular. The aspect ratio of the chamber may vary, such as tall (longer in the vertical dimension than the horizontal dimension) or short (longer in the horizontal dimension than the vertical dimension). The chamber orientation may be vertical, horizontal, or slanted. The chamber may be designed to have a flow pattern that is continuously stirred, plug flow, or flow distribution between these extremes.

The one or more UV light sources may be configured to expose ultraviolet light to at least 80%, at least 85%, at least 90%, at least 95%, or at least 99% of the total surface area within the chamber including its internal components. Internal components that are counted towards the chamber surface area may include a sparger, an impeller, an impeller shaft, a clean-in-place arm, an internal portion of a valve, and/or an internal portion of a sensor, for example. The total surface area of all internal components may be less than, about the same as, or greater than the surface area of the chamber walls facing the inside of the chamber.

The one or more UV light sources may be configured to expose ultraviolet light to at least 80%, at least 85%, at least 90%, at least 95%, or at least 99% of the total volume of the chamber. The total volume of the chamber is calculated from the nominal volume, based on the internal diameter and height, rather than the working volume.

In some embodiments, the one or more UV light sources each have a UV wavelength selected from about 100 nm to about 400 nm. In certain embodiments, the UV wavelength is selected from about 220 nm to about 300 nm, for at least one of the one or more UV light sources, such as for all of the UV light sources. In various embodiments, the UV wavelength is about, at least about, or at most about 100, 110, 120, 125, 130, 140, 150, 160, 170, 180, 190, 200, 210, 220, 230, 240, 250, 260, 270, 280, 290, 300, 310, 320, 330, 340, 350, 360, 370, 380, 390, or 400 nm, including all intervening ranges. In certain embodiments, the one or more UV light sources each have a UV wavelength selected from 125 nm to 400 nm.

In certain embodiments, the one or more UV light sources each have a UV wavelength selected from UV-C wavelengths, which for purposes of this specification are 200-290 nm. In certain embodiments, the one or more UV light sources each have a UV wavelength selected from UV-B wavelengths, which for purposes of this specification are 291-320 nm. In certain embodiments, the one or more UV light sources each have a UV wavelength selected from UV-A wavelengths, which for purposes of this specification are 321-400 nm.

The one or more UV light sources may each be selected from the group consisting of UV light-emitting diodes, UV mercury lamps, UV xenon lamps, and UV krypton lamps, for example. In certain embodiments, all UV light sources are UV light-emitting diodes (UV LEDs). A UV light source may be configured to generate pulsed UV light. For example, a pulsed-UV light source may be pulsed several times per second, with each pulse lasting between about 100 nanoseconds and 10 milliseconds.

In some embodiments, the bioreactor system contains an impeller situated within the chamber, wherein the impeller is preferably UV-sterilizable. In other embodiments, the chamber does not contain an impeller. When the chamber does not contain an impeller, the bioreactor is known as a bubble column. In a bubble column, there is no impeller; internal mixing is achieved via momentum transfer from the gas.

In some embodiments, the UV-sterilizable bioreactor system further comprises a filtration unit configured to filter an input material (e.g., a gas being fed to a sparger) before being fed into the chamber. In certain embodiments, the filtration unit is UV-sterilizable.

In some embodiments, the UV-sterilizable bioreactor system contains a bioreactor sensor situated within the chamber. The bioreactor sensor may be configured to detect or measure a bioreactor parameter selected from the group consisting of pH, temperature, oxygen, carbon dioxide, foaming, mixing, cell density, feed-substrate concentration, reaction-intermediate concentration, and product concentration, for example. There may be multiple bioreactor sensors situated within the chamber. In some embodiments, the bioreactor sensor is configured to transmit a wireless signal.

In some embodiments, the bioreactor sensor is UV-sterilizable. The bioreactor sensor may be configured with a UV optical waveguide for sterilizing the bioreactor sensor. The UV optical waveguide may be a UV optical fiber, for example.

In some embodiments, the bioreactor sensor is disposed within or through a probe port, wherein the probe port is UV-sterilizable. In some embodiments, the bioreactor sensor is contained in a UV-sterilizable housing that is situated within the chamber. The UV-sterilizable housing may contain a UV-transparent material, such as a UV-transparent polymer, a UV-transparent ceramic, a UV-transparent glass, or a combination thereof. A UV-transparent polymer may be selected from the group consisting of polyacrylates, silicones, fluoropolymers, and combinations thereof. An exemplary polyacrylate is poly(methyl methacrylate). Fluoropolymers may be selected from the group consisting of polyvinylidene fluoride, hexafluoropropylene-tetrafluoroethylene copolymers, perfluoroether-tetrafluoroethylene copolymers, poly(ethene-co-tetrafluoroethene), and combinations thereof. An exemplary UV-transparent ceramic is quartz. Exemplary UV-transparent glasses include fused silica, borosilicates, or a combination thereof. Silica or borosilicates may be doped to modify their UV transparency.

In some embodiments, the bioreactor system contains a clean-in-place arm situated within the chamber. A retractable clean-in-place arm may be utilized to clean the bioreactor before UV sterilization. In some embodiments, the clean-in-place arm is configured to facilitate preparation of the bioreactor system for fermentation.

The clean-in-place arm may be UV-sterilizable. In these embodiments, the clean-in-place arm may be exposed to a UV light source that also exposes UV light to the chamber walls or other components. Alternatively, or additionally, the clean-in-place arm may itself be configured with a UV sterilizer, arranged within or directly on the clean-in-place arm. In some embodiments, an array of UV LED light sources is internally mounted within the clean-in-place arm.

Additional components may be desirable for successful operation of the bioreactor system, such as (but not limited to) components for the introduction of reagents (e.g., conduits), for aeration (e.g., spargers), for agitation (e.g., impellers), for thermal management, for sensing, and for control.

In some embodiments, a port is a component in the bioreactor system, such that the port is used during operation without compromising the sterile operating environment. The port may be separated from the outside environment via pinch valves, diaphragm valves, or ball valves, for example. In certain embodiments, valves may be designed to sterilize reagents at the point-of-use during bioreactor operation. In some embodiments, a UV light source is arranged within or on a valve sterilizer. In certain embodiments, the UV light source is internally mounted within the valve sterilizer. The input that enters a valve typically comes through tubing from a source container. In some embodiments, the tubing or the source container are subjected to UV sterilization.

In some embodiments, aeration is accomplished using a gas-input component configured for introducing a gas into the chamber. The gas-input component may be a sparger, a membrane, or another means of effectively distributing a gas into the chamber. While it is possible to feed a gas through a port into the chamber, this usually leads to highly non-uniform gas concentrations within the chamber. In some embodiments, a gas input is sterilized via UV sterilization, filtration, or a combination thereof. In some embodiments, the gas-input component may have UV-light sources mounted within the component, such that sterile conditions are generated within the interior of the gas-input component.

An agitation component facilitates mixing of the internal constituents of the bioreactor chamber. In some embodiments, the agitation component is an impeller that achieves mixing by rotating around within the chamber, transferring kinetic energy to the chamber contents (usually in the turbulent flow, although can be laminar flow). The power to the impeller typically comes from an electric motor, i.e. powered by electricity, although in principle the impeller may be powered by magnetic induction or even compressed air. When an electrical motor is used, the motor is preferably mounted outside the chamber, and the motor may be sealed using a mechanical seal, a lip seal, or a magnetic seal, any of which may be UV-sterilized.

A thermal-management component may introduce or remove thermal energy (heat) from the bioreactor operating environment. In some embodiments, heat transfer may occur through the reactor walls, using a heat-transfer fluid, a steam jacket, heating coils, cooling coils, another thermal-management component, or a combination thereof. In some embodiments, internal baffles and/or internal coils are used to heat or cool the chamber contents. An external flow loop may be used to heat or cool the chamber contents.

Various components for sensing within the bioreactor system may be used, such as bioreactor sensors. In various embodiments, a bioreactor sensor detects or measures a bioreactor parameter selected from the group consisting of pH, temperature, dissolved oxygen, dissolved air, dissolved hydrogen, dissolved carbon monoxide, dissolved carbon dioxide, dissolved methane, foaming, mixing, cell density, feed-substrate concentration, reaction-intermediate concentration, product concentration, density, or weight, for example. The bioreactor sensor may measure various spectrophotometric characteristics. In some embodiments, the bioreactor sensor is configured to transmit a wireless signal to a computer for monitoring and control of the system.

Sensors may also be used for measuring a property within a product stream or a property of a sample. Product sensors or sample sensors may detect or measure a parameter selected from the group consisting of pH, temperature, dissolved oxygen, dissolved air, dissolved hydrogen, dissolved carbon monoxide, dissolved carbon dioxide, dissolved methane, cell density, product concentration, density, or weight, for example.

The measurement that is made with a sensor may be used to make process adjustments dynamically or in the future. The process adjustments may utilize well-known principles of process control, including proportional feedback control, proportional-integral-derivative (PID) feedback control, feedforward control, etc. A computer may be employed to automatically make adjustments to the process and system based on one or more measurements from sensors.

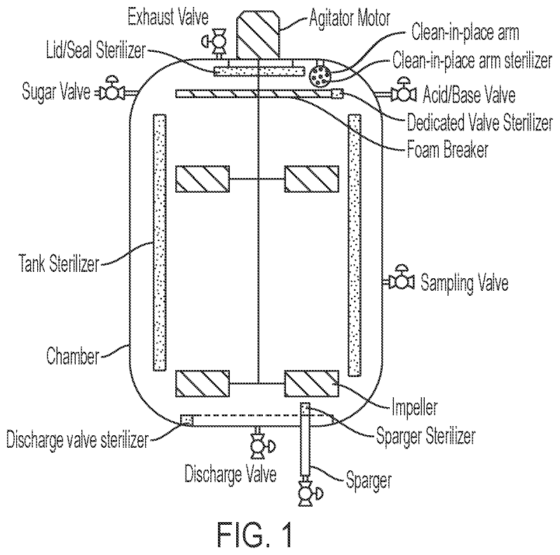

Some embodiments will now be described in reference to the accompanying drawings of FIGS. 1 to 5, which are intended to illustrate various examples of the invention and shall not be construed to limit the invention.

FIG. 1 is a schematic diagram (side view) of a UV-sterilizable bioreactor system, in some embodiments of the invention. In FIG. 1, there is a chamber configured to carry out a fermentation reaction, a sparger configured to introduce a gas into the chamber volume, and tank sterilizers (UV light sources) configured to expose UV light to surfaces within the chamber volume. FIG. 1 shows two tank sterilizers, it being understood that the number of tank sterilizers shown is merely exemplary. The sparger is equipped with a sparger sterilizer configured to expose UV light to the sparger itself. There is a sugar valve for introducing a sugar substrate and media components. There is acid/base valve for introducing acids, bases, and/or buffers to the chamber. The acid/base valve is equipped with a dedicated valve sterilizer configured to expose UV light to the valve itself. A sampling valve allows for extracting samples before, during, or after fermentation. A discharge valve allows for discharging the fermentation broth during or after fermentation. The discharge valve is equipped with a discharge valve sterilizer configured to expose UV light to the discharge valve. An exhaust valve allows for discharging gas during or after fermentation. There is a lid/seal sterilizer configured to expose UV light to the lid and seal at the top of the chamber, near the agitator motor. There is a clean-in-place arm at or near the top of the chamber. The clean-in-place arm is equipped with a clean-in-place arm sterilizer configured to expose UV light to the clean-in-place arm. The diagram of FIG. 1 depicts four impellers, it being understood that the number of impellers shown is merely exemplary. A foam breaker is configured near the top of the chamber. Note that FIG. 1 explicitly shows tank sterilizers, a sparger sterilizer, an acid/base valve sterilizer, a UV clean-in-place arm sterilizer, a lid/seal sterilizer, a discharger valve sterilizer; in other embodiments of the invention, fewer UV sterilizers are employed (e.g., only tank sterilizers), or additional UV sterilizers are employed (e.g., a sugar valve sterilizer is also used).

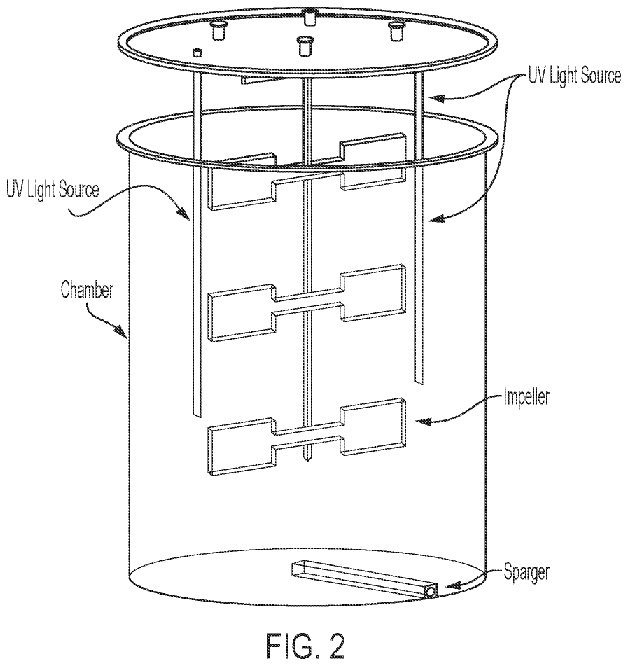

FIG. 2 is a schematic diagram (isometric view) of a UV-sterilizable bioreactor system, in some embodiments of the invention. In FIG. 2, there is a chamber configured to carry out a fermentation reaction, a sparger configured to introduce a gas into the chamber volume, and UV light sources configured to expose UV light to surfaces within the chamber volume. FIG. 2 shows two UV light sources, it being understood that the number of UV light sources shown is merely exemplary. The diagram of FIG. 1 depicts six impellers, it being understood that the number of impellers shown is merely exemplary.

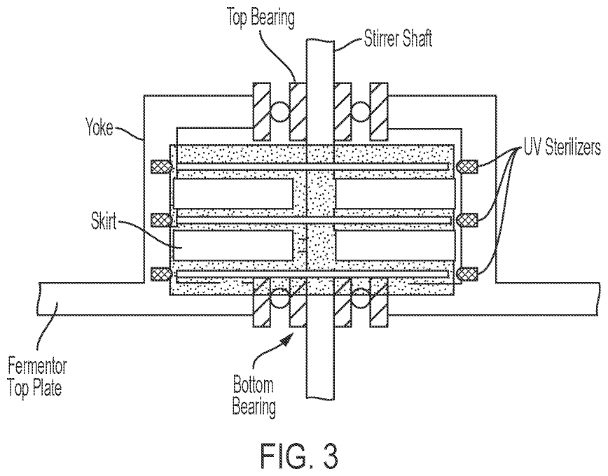

FIG. 3 is a schematic diagram (side view) of a UV-sterilizable bioreactor top with stirrer seal, in some embodiments of the invention. The UV-sterilizable bioreactor top is designed to maintain a sterile boundary with the environment. In FIG. 3, a fermentor top plate is configured such that an agitator stirrer shaft passes through the plate while maintaining a sterile barrier. A bottom bearing for a stirrer shaft is fitted in the center of the fermentor top plate. A top bearing for the stirrer shaft is fitted in a yoke directly above the fermentor top plate. The bearings are pressed into housings within the yoke. A skirt provides a shield to prevent airborne contaminants from settling on the bottom bearing and eventually infiltrating into the chamber. There are multiple UV sterilizers (UV light sources) configured to expose UV light through the assembly.

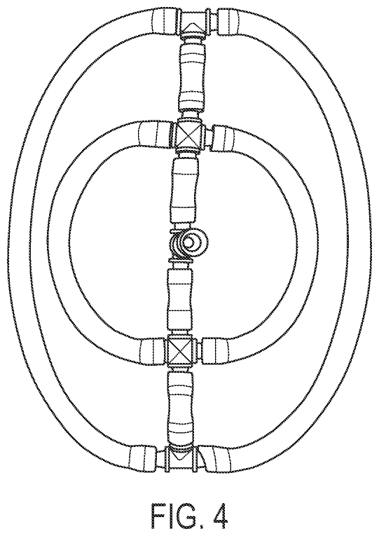

FIG. 4 is a schematic diagram (top view) of a UV-sterilizable sparger, in some embodiments of the invention. The sparger may be UV-sterilized by being exposed to a UV light source that is external to the sparger. The sparger may be UV-sterilizable by incorporating a UV light source within the sparger itself. The sparger may be fabricated from a UV-transparent material, such as a UV-transparent polymer, a UV-transparent ceramic, a UV-transparent glass, or a combination thereof. In FIG. 4, a gas such as air or oxygen enters through the top port shown in the middle of the diagram from a pipe or tube that is not shown. The gas circulates around the curved tubes, shown as having an inner circle and an outer circle. The gas is distributed out into the chamber through small holes (not shown) in the tubes.

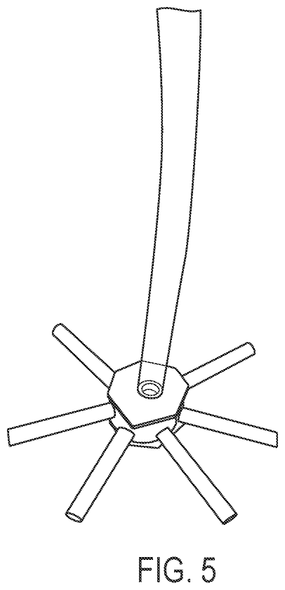

FIG. 5 is a schematic diagram (isometric view) of another UV-sterilizable sparger, in some embodiments of the invention. The sparger may be UV-sterilized by being exposed to a UV light source that is external to the sparger. The sparger may be UV-sterilizable by incorporating a UV light source within the sparger itself. The sparger may be fabricated from a UV-transparent material, such as a UV-transparent polymer, a UV-transparent ceramic, a UV-transparent glass, or a combination thereof. In FIG. 5, a gas such as air or oxygen enters through the top tube (hub) and is distributed out into a chamber through outlets contained in each of the spokes pointed outward. The gas can also be distributed out into the chamber through small holes in the tubes, for example. The drawing depicts 6 spokes (an additional spoke may be implied but hidden in view from the hub).

Some variations provide a method of cleaning and sterilizing a bioreactor, the method comprising:
(i) providing a UV-sterilizable bioreactor system comprising: (a) a chamber configured to carry out a reaction; (b) a component configured for introducing a gas into the chamber; and (c) one or more UV light sources configured to expose ultraviolet light to surfaces within the chamber;
(ii) cleaning the chamber; and
(iii) exposing the chamber to UV radiation to sterilize the chamber.

In some methods, the UV-sterilizable bioreactor system further comprises one or more conduits configured to feed an input material into the chamber and/or to withdraw an output material out of the chamber. A conduit may be a valve, an inlet, or an outlet. In certain methods, at least one of the conduits is UV-sterilizable. A conduit may be UV-sterilizable when it is exposed to a UV light source that is external to the conduit. Alternatively, or additionally, a conduit may have built-in UV light source (e.g., UV LEDs, built onto or into the conduit, such that it is UV-sterilized when desired.

In some methods, one or more UV light sources, such as all of the UV light sources, are situated within the chamber. In certain methods, one or more UV light sources, such as all of the UV light sources, are permanently situated within the chamber. In certain methods, one or more UV light sources, such as all of the UV light sources, are reversibly situated within the chamber.

In some methods, one or more UV light sources, such as all of the UV light sources, are situated within UV-transparent wells that are disposed within the chamber.

In some methods, at least some of the one or more UV light sources are external to the chamber. In certain methods, all of the one or more UV light sources are external to the chamber.

In some methods, the chamber has walls fabricated from a metal, a metal alloy, a polymer, a ceramic, a composite material, glass, concrete, or a combination thereof.

In some methods, the chamber has UV-transparent chamber walls containing a UV-transparent material. UV-transparent materials are discussed earlier in this specification.

In some methods, the chamber has UV-reflective chamber walls containing, or internally coated with, a UV-reflective material.

In certain methods, the chamber is configured with a chamber top that is not sealed from the environment, wherein the chamber top is UV-sterilizable to form a sterile barrier with the environment.

In some methods, an impeller is situated within the chamber, wherein the impeller is UV-sterilizable.

In some methods, the UV-sterilizable bioreactor system further comprises a filtration unit configured to filter an input material before being fed into the chamber. In certain methods, the filtration unit is UV-sterilizable.

In some methods, a bioreactor sensor is situated within the chamber. A bioreactor sensor may be configured to detect or measure a bioreactor parameter selected from the group consisting of pH, temperature, oxygen, carbon dioxide, foaming, mixing, cell density, feed-substrate concentration, reaction-intermediate concentration, and product concentration. A bioreactor sensor may be configured with an optical microscope, camera, or IR scope to take a picture of the mass distribution and/or the heat distribution at certain locations in the bioreactor, such as near the sparger. The bioreactor sensor may be configured to transmit a wireless signal during or after sensor measurement. During operation, the bioreactor sensor may actually detect or measure a bioreactor parameter. Control strategies may be used to adjust bioreactor parameters; for example, if dissolved oxygen is measured to be too low, a higher sparging rate of air or O₂ may be fed into the bioreactor chamber.

In some methods, the bioreactor sensor is UV-sterilizable. The bioreactor sensor may be configured with a UV optical waveguide for sterilizing the bioreactor sensor. Alternatively, or additionally, the bioreactor sensor is contained in a UV-sterilizable housing that is situated within the chamber. The UV-sterilizable housing may contain a UV-transparent material. Alternatively, or additionally, the bioreactor sensor may be disposed within or through a probe port, wherein the probe port is UV-sterilizable.

In some methods, the bioreactor contains a clean-in-place arm situated within the chamber, wherein the clean-in-place arm is UV-sterilizable. The clean-in-place arm may be UV-sterilized during step (iii), or in a different step.

In some methods, step (ii) utilizes a cleaning agent selected from the group consisting of hot water, an alkaline detergent, sodium hydroxide, sodium percarbonate, an acidic detergent, phosphoric acid, peracetic acid, isopropanol, ethanol, sodium hypochlorite, hydrogen peroxide, ethylene oxide, chlorine dioxide, ozone, formaldehyde, peracetic acid, glutaraldehyde, and combinations thereof. Other cleaning agents may be utilized instead of, or in addition to, these chemicals. The cleaning agent may be a liquid, a vapor, a gas, or a combination thereof (e.g., a liquid/vapor mixture, or a liquid followed by a gas, etc.).

In some methods, step (iii) utilizes a sterilization energy per unit total area in the chamber of about 50, 100, 150, 200, 250, 300, 400, 500, 600, 700, 800, 900, 1000, 1500, 2000, 3000, 4000, or 5000 mJ/cm², or more, including all intervening ranges. The sterilization energy per unit total area is also known as the light intensity. The light intensity may be constant, or it may vary over time.

In some methods, step (iii) utilizes a total UV power output from about 0.5 mW/cm² to about 1000 mW/cm². The total UV power may be about, at least about, or at most about 0.5, 1, 2, 3, 4, 5, 10, 50, 100, 200, 300, 400, 500, 600, 700, 800, 900, 1000 mW/cm², including all intervening ranges. The total UV power output may be constant, or it may vary over time.

In some methods, step (iii) utilizes a total UV power capacity from about 0.1 W/m³ to about 5000 W/m³. The total UV power capacity is calculated as input UV power divided by volume of the chamber. The total UV power capacity may be about, or at most about 5000, 4000, 3000, 2000, 1000, 500, 400, 300, 200, 100, 50, 40, 30, 25, 20, 15, 10, 9, 8, 7, 6, 5, 4, 3, 2, 1, 0.5, 0.4, 0.3, 0.2, or 0.1 W/m³, including all intervening ranges.

In some methods, step (iii) utilizes a UV sterilization time from about 1 minute to about 24 hours. The UV sterilization time is the time that the UV light source is illuminated. In various embodiments, the UV sterilization time is about, at least about, or at most about 1 minute, 2 minutes, 3 minutes, 4 minutes, 5 minutes, 10 minutes, 15 minutes, 20 minutes, 25 minutes, 30 minutes, 45 minutes, 1 hour, 2 hours, 3 hours, 4 hours, 5 hours, 6 hours, 7 hours, 8 hours, 12 hours, 16 hours, 20 hours, or 24 hours, including all intervening ranges.

In some methods, step (iii) is conducted at a sterilization temperature from about 10° C. to about 95° C. In various embodiments, the sterilization temperature is about, at least about, or at most about 10° C., 15° C., 20° C., 25° C., 30° C., 35° C., 40° C., 45° C., 50° C., 55° C., 60° C., 65° C., 70° C., 75° C., 80° C., 85° C., 90° C., or 95° C., including any intervening range. In some embodiments, the sterilization temperature is not controlled and is at or about the ambient temperature, such as about 25° C. In other embodiments, it is desirable to increase the temperature to assist in the sterilization.

In typical methods, step (iii) is conducted in an atmosphere that consists essentially of air, which may be dry air or humid air. In typical methods, step (iii) is conducted in an atmosphere that does not contain steam, although water vapor may be present, due to humidity in air, for example.

In some methods, step (iii) is conducted in an inert-gas atmosphere, such as under CO₂, N₂, Ar, or a combination thereof. When step (iii) is carried out with air present in the chamber, the air contains N₂ that is inert and O₂ that may or may not be inert.

In certain methods, step (iii) is conducted in an atmosphere that contains a sterilization-enhancing vapor or gas, such as ethylene oxide, chlorine dioxide, hydrogen peroxide, ozone, formaldehyde, peracetic acid, or glutaraldehyde. Ozone may be generated in situ from photolysis of oxygen (e.g., from air), creating O₃ from O₂, such as when one or more UV wavelengths in the 160-240 nm range are used for the UV sterilization.

In some methods, step (iii) exposes the UV radiation to at least 80% of total surface area within the chamber including its internal components. Step (iii) may expose the UV radiation to at least 85%, at least 90%, at least 95%, or at least 99% (including 100%) of total surface area within the chamber including its internal components. The “internal components” of the chamber are those components, or portions thereof, that are inside the boundary with the environment.

In some methods, step (iii) exposes the UV radiation to at least 80% of total volume of the chamber. Step (iii) may expose the UV radiation to at least 85%, at least 90%, at least 95%, or at least 99% (including 100%) of total volume of the chamber.

In some methods, step (iii) utilizes a UV wavelength selected from about 100 nm to about 400 nm, such as a UV wavelength selected from about 220 nm to about 300 nm. In various methods, step (iii) utilizes one or more UV wavelengths selected from about, at least about, or at most about 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 nanometers, including any intervening ranges (e.g., 200-300 nm), and inclusive of each 1-nanometer increment within each sub-range (e.g., including 227-304 nm, 188-242 nm, etc.). In some embodiments, such as (but not limited to) those utilizing pulsed light, multiple radiation wavelengths may be provided by the light source, some of which fall in the 100-400 nm range and some of which are higher than 400 nm, such as in the visible or infrared range in the electromagnetic spectrum.

With respect to wavelength, while 100-400 nm is a preferred range of radiation wavelengths, it will be recognized that higher wavelengths can also be effective in certain embodiments, depending on the specific adventitious microorganism and the radiation parameters other than wavelength (e.g., time, light intensity, total power, or absorbed energy). Wavelengths in the visible band (about 400-700 nm) and/or infrared band (about 700 nm-1 mm) may be capable of inactivating adventitious microorganisms, such as by inducing DNA lesions.

Some variations generally provide a radiation-sterilizable bioreactor system comprising:
(a) a chamber configured to carry out a reaction;
(b) a component configured for introducing a gas into the chamber; and
(c) one or more light sources configured to expose radiation to surfaces within the chamber, wherein the radiation has a wavelength longer than 1 nm but less than 1 mm.

Some variations generally provide a method of cleaning and sterilizing a bioreactor, the method comprising:
(i) providing a radiation-sterilizable bioreactor system comprising: (a) a chamber configured to carry out a reaction; (b) a component configured for introducing a gas into the chamber; and (c) one or more light sources configured to expose radiation to surfaces within the chamber;
(ii) cleaning the chamber; and
(iii) exposing the chamber to radiation to sterilize the chamber, wherein the radiation has a wavelength longer than 1 nm but less than 1 mm.

In preferred embodiments of a radiation-sterilizable bioreactor system and a method of cleaning and sterilizing a bioreactor, the one or more light sources are configured to expose non-ionizing UV radiation, visible light, IR radiation, or a combination thereof to the surfaces within the chamber. X-rays and gamma rays are examples of ionizing radiation; therefore, X-rays or gamma rays are not within the scope of non-ionizing UV radiation. In preferred methods, the light sources are UV light sources, which may be selected from the group consisting of UV light-emitting diodes, UV mercury lamps, UV xenon lamps, and UV krypton lamps.

In various methods, step (iii) is effective to reach a 4-log reduction in adventitious microorganisms present prior to step (iii). In this specification, “log” refers to the common base-10 logarithm. Thus, a 4-log reduction is a 99.99% reduction in population of adventitious microorganisms because the fraction of adventitious microorganisms remaining is 10⁻⁴, and log 10⁻⁴=−4. In some methods, step (iii) is effective to reach a 6-log reduction in adventitious microorganisms present prior to step (iii). In certain methods, step (iii) is effective to reach a 8-log reduction in adventitious microorganisms present prior to step (iii). In specific methods, step (iii) is effective to reach a 10-log reduction in adventitious microorganisms present prior to step (iii). In various embodiments, step (iii) is effective to reach a reduction in adventitious microorganisms, present initially prior to step (iii), that is about, or at least about, 3, 4, 5, 6, 7, 8, 9, 10, 11, or 12 logs. In certain embodiments, step (iii) is effective to reach a complete elimination of adventitious microorganisms, present initially prior to step (iii), i.e. 100% reduction which may be referred to as achieving aseptic conditions. Generally speaking, different microorganisms have different vulnerabilities to UV light. When there are multiple species of adventitious microorganisms present, the kill rates of the different species may differ.

In bioreactor facilities, adventitious microorganisms can include spore-forming gram-positive or gram-negative rods; non-spore-forming bacteria; fungal contaminations; or cocci, for example. The microorganism for the desired fermentation may or may not be more LTV-tolerant than adventitious microorganisms. If the adventitious microorganisms are especially UV-tolerant and survive the UV sterilization, this poses no problem for the eventual fermentation in the bioreactor.

The method may further comprise, after step (iii), introducing gas to the chamber. The gas may be air, oxygen, syngas, hydrogen, carbon monoxide, methane, natural gas, or a combination thereof, for example. The gas may be a mixture of O₂ and N₂ in various concentrations of O₂, above or below 21 vol % O₂. The gas may be a reactant, such as in aerobic or microaerobic fermentation, or may be a catalyst, promoter, reaction-rate moderator, or other reaction agent. The gas may be first sterilized, prior to being fed to the chamber, via filtration, exposure to UV light, or a combination thereof (e.g., by filtering through a UV-sterilized filter).

To carry out a desired fermentation, there will generally be several inputs such as, but not limited to, media, a feedstock acting as a carbon source, acid input, base input, anti-foaming agent, buffer, vitamins, gaseous input, or any bolus of materials intended to influence the biochemical process.

The fermentation may be conducted at any suitable fermentation temperature, such as from about 10° C. to about 60° C. In various embodiments, the fermentation temperature is about, at least about, or at most about 10° C., 15° C., 20° C., 25° C., 30° C., 35° C., 40° C., 45° C., 50° C., 55° C., or 60° C., including any intervening range. Multiple fermentation temperatures may be used, such as an initial temperature during a cell-growth phase and a second temperature during a production phase.

Fermentation temperature may be monitored and controlled via thermal management. In some embodiments, thermal management is achieved via heat exchange through a chamber wall. In some embodiments, thermal management is achieved via heat exchange through internal cooling baffles. In some embodiments, thermal management is achieved via heat exchange through an external cooling loop.

The fermentation may be conducted at any suitable fermentation pH, such as from about 2 to about 12. In various embodiments, the fermentation pH as about, at least about, or at most about 2, 3, 4, 5, 6, 7, 8, 9, 10, 11, or 12, including any intervening range. Multiple fermentation pH values may be used, such as an initial pH during a first-product phase and a second pH during a second-product phase.

The fermentation may be conducted at any suitable fermentation pressure, such as from about 0.1 bar (vacuum) to about 20 bar. In various embodiments, the fermentation pressure is about, at least about, or at most about 0.1, 0.2, 0.3, 0.4, 0.5, 0.6, 0.7, 0.8, 0.9, 0.95, 1.0, 1.05, 1.1, 1.2, 1.3, 1.4, 1.5, 1.6, 1.7, 1.8, 1.9, 2, 3, 4, 5, 6, 7, 8, 9, or 10 bar, including any intervening range. Microbial growth rates and product formation can be improved at higher pressures, such as when fermenting syngas, because a higher pressure increases the driving force for mass transfer of H₂ and CO into the liquid phase that contains the microorganism cells.

The fermentation may be conducted at any suitable gas (e.g., O₂) concentration, such as from about 0.1 mg/L to about 100 mg/L (milligrams gas per liter of liquid present). In various embodiments, the gas concentration is about, at least about, or at most about 0.1, 0.5, 1, 5, 10, 15, 20, 25, 30, 35, 40, 45, 50, 60, 70, 80, 90, or 100 mg/L, including any intervening range. Multiple fermentation gas concentrations values may be used, such as an initial gas concentration during a cell-growth phase and a second gas concentration during a production phase.

The fermentation may be conducted at any suitable cell concentration, such as from about 0.1 g/L to about 200 g/L (grams of cells of desired biocatalyst per liter of liquid present). In various embodiments, the cell concentration is about, at least about, or at most about 0, 0.1, 0.5, 1, 1.5, 2, 3, 4, 5, 10, 25, 50, 100, 150, or 200 g/L, including any intervening range. In some embodiments, the cell concentration is roughly constant during fermentation. In other embodiments, there is a cell-growth phase during which active cells of the biocatalyst are generated, followed by a production phase wherein the cells make a product (e.g., organic acids, enzymes, etc.) that is released from the cells. In still other embodiments, the cells are the desired product, the cell concentration may increase during the entire fermentation.

The fermentation may be conducted for any suitable fermentation time, such as from about 8 hours to about 350 hours, such as about, at least about, or at most about 8, 12, 16, 24, 36, 48, 72, or 96 hours, including any intervening range. For a batch or fed-batch fermentation, the fermentation time starts when all fermentation components are present at the fermentation temperature, pressure, pH, and gas concentration, and the fermentation time ends when one or more parameters are adjusted to stop the fermentation and/or the bioreactor is emptied to recover the product. For a continuous fermentation, the fermentation time is the residence time, which is the inverse of the dilution rate.

The bioreactor may be operated in batch, in semi-batch, in fed-batch, semi-continuously, continuously, or a combination or hybrid thereof.

When the bioreactor is operated in batch mode, typically step (ii) and step (iii) are each performed after every batch. In other embodiments, step (ii) may be performed after some batches but not after other batches. In these or other embodiments, step (iii) may be performed after some batches but not after other batches. Various protocols may be used, and measurements may be made (e.g., composition or biological analysis) to determine whether to conduct step (ii) and/or step (iii) after a given batch.

When the bioreactor is operated continuously, typically step (ii) is performed to clean the chamber, followed by step (iii) being performed to sterilize the chamber, which is then followed by a period of continuous operation of the bioreactor to produce a product. During such continuous operation, various inputs or outputs may be sterilized, such as (but not limited to) substrates (e.g., sugar), gases (e.g., air), vitamins, minerals, acids, bases, buffers, antifoam, samples, or products, using UV light sources. Such inputs or outputs may be sterilized intermittently or, more preferably, continuously.

Generally speaking, the principles of the invention can be applied to a wide variety of commercial processes and products, including (but not limited to) industrial chemicals, biochemicals, biofuels, pharmaceuticals, nutraceuticals, vitamins, food ingredients, protein products, enzymes, and cells.

In some cases, sucrose and oxygen are combined to synthesize riboflavin. In some cases, glucose and oxygen are combined to synthesize hyaluronic acid. In some cases, molasses and oxygen are combined to synthesize citric acid. In some cases, complex media and oxygen are combined to produce bovine cells. In some cases, gaseous carbon monoxide or carbon dioxide and gaseous hydrogen are combined to produce ethanol. In some cases, formic acid and oxygen are combined to produce lactic acid. In some cases, glycerol and oxygen are combined to produce glycolic acid. In some embodiments, methanol and oxygen are combined to produce microbial biomass. In some embodiments, methane and oxygen are combined to produce organic acids. In some cases, feedstock and oxygen are combined to produce multicellular organisms. In some embodiments, carbon feedstock and oxygen are combined to produce filamentous fungi. In some embodiments, waste carbon (e.g., waste oils) and oxygen are combined to produce polyhydroxyalkanoates. In some embodiments, glucose and oxygen are combined to produce insulin. In some embodiments, sucrose and oxygen are combined to make farnesene.

In preferred methods, the chamber maintains a sterile boundary with the environment. A sterile boundary may be maintained using a gas-tight seal. Alternatively, or additionally, a sterile boundary may be maintained by UV-sterilizing an open region of the chamber (e.g., an open chamber top) to form a type of virtual, rather than physical, sterile barrier with the environment.

In certain embodiments, after step (iii) and during operation to produce a fermentation product, one or more UV light sources are still used. The UV light sources used during bioreactor operation may be less than all the UV light sources used for UV sterilization in step (ii). For example, UV LEDs may be continuously or intermittently utilized during bioreactor operation to prevent the ingress of adventitious microorganisms. It is conceptually possible to use all UV light sources utilized in step (ii) also during bioreactor operation, if the active (desired) biocatalyst is more UV-tolerant than adventitious microorganisms, or if some level of UV exposure to the biocatalyst is desired, such as during a mutagenesis campaign, for example.

Following production of a fermentation product, the product will typically be present in a dilute solution or broth. Product recovery may be performed using known techniques, such as (but not limited to) evaporation, distillation, centrifuge, liquid-liquid extraction, to generate a concentrated form of a desired product. In certain embodiments, no product concentration is necessary because the as-is fermentation broth is the product which may be stored, shipped, or used elsewhere at a plant site.

In some embodiments, one or more materials recovered from a dilute solution or broth is recovered and recycled for reuse at a plant site or an adjacent site, for example. For example, water may be recovered and reused, to improve the water balance. Vitamins and minerals may be recovered and reused in another fermentation. Any recovered materials may be subjected to UV sterilization prior to reuse, or UV sterilization at the point of re-entry into a process, such as via UV-sterilized filters. In some embodiments, biological material is inactivated upon removal from the bioreactor system via UV sterilization.

In this detailed description, reference has been made to multiple embodiments which show by way of illustration specific exemplary embodiments of the invention. These embodiments are described in sufficient detail to enable those skilled in the art to practice the invention, and it is to be understood that modifications to the various disclosed embodiments may be made by a skilled artisan.

Where methods and steps described above indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

All publications, patents, and patent applications cited in this specification are herein incorporated by reference in their entirety as if each publication, patent, or patent application were specifically and individually put forth herein. This specification hereby incorporates by reference Stanbury et al., Principles of Fermentation Technology, 3^{rd} Edition, Elsevier, 2017.

The embodiments and variations described above should provide an indication of the utility and versatility of the present invention. Other embodiments that do not provide all of the features and advantages set forth herein may also be utilized, without departing from the spirit and scope of the present invention. Such modifications and variations are considered to be within the scope of the invention defined by the claims.

### Example

A 4 L glass vessel with a custom headplate is manufactured. The system is cleaned using Alcojet® detergent and water, after which it is dried. The head plate and vessel are combined using a silicone gasket to achieve a hermetic seal.

UV sterilization is performed in the glass vessel, as follows. Three 11 W UV-C bulbs are inserted into UV-transparent wells that are securely affixed within the acrylic headplate. These bulbs are powered on for 60 minutes, during which time the hermetic chamber seal is maintained with the external environment.

2 L of sterile trypticase soy broth (TSB) media are then introduced into the bioreactor volume using a sterile interface present on the headplate. 1 vessel volume per minute (VVM) of air is introduced into the TSB media through a sterilizing 0.22-micron filter within the bioreactor volume for 5 days. Samples are removed from the bioreactor volume in a sterile manner during this period.

The optical density at 600 nm (OD600) is used to characterize microbial growth within the bioreactor volume. A measurement of OD600=0 is taken at day 0, day 1, day 2, day 3, day 4, and day 5. Additionally, samples are plated on both TSB and fluid thioglycolate plates which were then incubated at respective temperatures of 22° C. and 32° C. for 2 weeks. No observable growth is detected.

### Comparative Example

A 4 L glass vessel with a custom headplate is manufactured. The system is cleaned using Alcojet® detergent and water, after which it is dried. The head plate and vessel are combined using a silicone gasket to achieve a hermetic seal. No UV sterilization is performed on or in the glass vessel.

2 L of sterile trypticase soy broth (TSB) media are then introduced into the bioreactor volume using a sterile interface present on the headplate. 1 vessel volume per minute (VVM) of air is introduced into the TSB media through a sterilizing 0.22-micron filter within the bioreactor volume for 5 days. Samples are removed from the bioreactor volume in a sterile manner during this period.

The optical density at 600 nm (OD600) is used to characterize microbial growth within the bioreactor volume. The OD600 measurement is significantly greater than 0 after day 1, after which point the experiment can be terminated. Additionally, samples are plated on both TSB and fluid thioglycolate plates which are then incubated at respective temperatures of 22° C. and 32° C. for 2 weeks. Significant observable growth is detected.

What is claimed is:

1. A UV-sterilizable bioreactor system comprising:
(a) a chamber configured to carry out a reaction;
(b) a component configured for introducing a gas into said chamber; and
(c) one or more UV light sources configured to expose ultraviolet light to surfaces within said chamber.

2. The UV-sterilizable bioreactor system of claim 1, wherein said chamber is configured to maintain a sterile boundary with the environment.

3. The UV-sterilizable bioreactor system of claim 1, wherein said component is a gas sparger.

4. The UV-sterilizable bioreactor system of claim 1, wherein said component is a membrane.

5. The UV-sterilizable bioreactor system of claim 1, wherein said component is UV-sterilizable.

6. The UV-sterilizable bioreactor system of claim 1, wherein said component contains a UV-transparent material.

7. The UV-sterilizable bioreactor system of claim 6, wherein said UV-transparent material contains a UV-transparent polymer, a UV-transparent ceramic, a UV-transparent glass, or a combination thereof.

8. The UV-sterilizable bioreactor system of claim 7, wherein said UV-transparent polymer is selected from the group consisting of polyacrylates, silicones, fluoropolymers, and combinations thereof.

9. The UV-sterilizable bioreactor system of claim 8, wherein said fluoropolymers are selected from the group consisting of polyvinylidene fluoride, polytetrafluoroethylene, perfluoroalkoxy alkanes, and combinations thereof.

10. The UV-sterilizable bioreactor system of claim 7, wherein said UV-transparent ceramic is selected from the group consisting of quartz, fused silica, borosilicates, and combinations thereof.

11. The UV-sterilizable bioreactor system of claim 1, wherein said gas is air.

12. The UV-sterilizable bioreactor system of claim 1, wherein said gas is oxygen.

13. The UV-sterilizable bioreactor system of claim 1, wherein said gas is syngas, hydrogen, carbon monoxide, methane, natural gas, or a combination thereof.

14. The UV-sterilizable bioreactor system of claim 1, wherein said UV-sterilizable bioreactor system further comprises one or more conduits configured to feed an input material into said chamber and/or to withdraw an output material out of said chamber.

15. The UV-sterilizable bioreactor system of claim 14, wherein at least one of said one or more conduits is UV-sterilizable.

16. The UV-sterilizable bioreactor system of claim 1, wherein said one or more UV light sources are situated within said chamber.

17. The UV-sterilizable bioreactor system of claim 1, wherein said one or more UV light sources are situated within UV-transparent wells that are disposed within said chamber.

18. The UV-sterilizable bioreactor system of claim 1, wherein said one or more UV light sources are permanently situated within said chamber.

19. The UV-sterilizable bioreactor system of claim 1, wherein said one or more UV light sources are reversibly situated within said chamber.

20. The UV-sterilizable bioreactor system of claim 1, wherein at least some of said one or more UV light sources are external to said chamber.

21. The UV-sterilizable bioreactor system of claim 1, wherein all of said one or more UV light sources are external to said chamber.

22. The UV-sterilizable bioreactor system of claim 1, wherein said chamber has walls fabricated from a metal, a metal alloy, a polymer, a ceramic, a composite material, glass, concrete, or a combination thereof.

23. The UV-sterilizable bioreactor system of claim 1, wherein said chamber has UV-transparent chamber walls containing a UV-transparent material.

24. The UV-sterilizable bioreactor system of claim 1, wherein said chamber has UV-reflective chamber walls containing, or internally coated with, a UV-reflective material.

25. The UV-sterilizable bioreactor system of claim 1, wherein said chamber is configured with a chamber top that is not sealed from the environment, and wherein said chamber top is UV-sterilizable to form a sterile barrier with said environment.

26. The UV-sterilizable bioreactor system of claim 1, wherein said one or more UV light sources are configured to expose ultraviolet light to at least 80% of total surface area within said chamber including its internal components.

27. The UV-sterilizable bioreactor system of claim 1, wherein said one or more UV light sources are configured to expose ultraviolet light to at least 80% of total volume of said chamber.

28. The UV-sterilizable bioreactor system of claim 1, wherein said one or more UV light sources each have a UV wavelength selected from about 100 nm to about 400 nm.

29. The UV-sterilizable bioreactor system of claim 1, wherein said one or more UV light sources are selected from the group consisting of UV light-emitting diodes, UV mercury lamps, UV xenon lamps, and UV krypton lamps.

30. The UV-sterilizable bioreactor system of claim 1, wherein said bioreactor system contains an impeller situated within said chamber, and wherein said impeller is UV-sterilizable.

31. The UV-sterilizable bioreactor system of claim 1, wherein said chamber does not contain an impeller.

32. The UV-sterilizable bioreactor system of claim 1, wherein said UV-sterilizable bioreactor system further comprises a filtration unit configured to filter an input material before being fed into said chamber.

33. The UV-sterilizable bioreactor system of claim 32, wherein said filtration unit is UV-sterilizable.

34. The UV-sterilizable bioreactor system of claim 1, wherein said UV-sterilizable bioreactor system contains a bioreactor sensor situated within said chamber, and wherein said bioreactor sensor is UV-sterilizable.

35. The UV-sterilizable bioreactor system of claim 34, wherein said bioreactor sensor is configured to detect or measure a bioreactor parameter selected from the group consisting of pH, temperature, oxygen, carbon dioxide, foaming, mixing, cell density, feed-substrate concentration, reaction-intermediate concentration, and product concentration.

36. The UV-sterilizable bioreactor system of claim 34, wherein said bioreactor sensor is contained in a UV-sterilizable housing that is situated within said chamber.

37. The UV-sterilizable bioreactor system of claim 34, wherein said bioreactor sensor is disposed within or through a probe port, and wherein said probe port is UV-sterilizable.

38. The UV-sterilizable bioreactor system of claim 1, wherein said bioreactor system contains a clean-in-place arm situated within said chamber.

39. The UV-sterilizable bioreactor system of claim 38, wherein said clean-in-place arm is UV-sterilizable.
